SEQUENCE LISTING (41 sequences)

SEQ ID NO: 1 - protein, 23 aa, Artificial
  Synthetic peptide
RHWTALGPAPTHTCADLNYPLLS

SEQ ID NO: 2 - protein, 37 aa, Artificial
  Synthetic peptide
STKTLGRPLHGPAGPVEGGALAGVAEDADLVTAVSGR

SEQ ID NO: 3 - protein, 37 aa, Artificial
  Synthetic peptide
YHCKREDLTDRDATCALRQPPQAVRGLGPRVTAVSGR

SEQ ID NO: 4 - protein, 15 aa, Artificial
  Synthetic peptide
RRAEITHPGMMLASG

SEQ ID NO: 5 - protein, 36 aa, Artificial
  Synthetic peptide
HNPFAIHRWECCTPALRALVGPDVQQLPVLTAVSGR

SEQ ID NO: 6 - protein, 36 aa, Artificial
  Synthetic peptide
VVHLLALPALLAREVGPPQLGSLDPLPQRVTAVSGR

SEQ ID NO: 7 - protein, 40 aa, Artificial
  Synthetic peptide
TALQVAAALDVGPLQGRQVQLGERLLPAREVTAVSCGRSS

SEQ ID NO: 8 - protein, 29 aa, Artificial
  Synthetic peptide
NVGTCTSSPARCGWPRRRTSCAALAGLLV

SEQ ID NO: 9 - protein, 16 aa, Artificial
  Synthetic peptide
KADILPEMNSMRADRM

SEQ ID NO: 10 - protein, 37 aa, Artificial
  Synthetic peptide
WERGRRVGAQVRHARHLVARVLDGAGHQARLTAVNGP

SEQ ID NO: 11 - protein, 16 aa, Artificial
  FVIII fragment
LVKDLNSGLIGALLVC

SEQ ID NO: 12 - protein, 19 aa, Artificial
  FVIII fragment
DLASGLIGPLLICYKESVD

SEQ ID NO: 13 - protein, 13 aa, Artificial
  FVIII fragment
RNRGMTALLKVSS

SEQ ID NO: 14 - protein, 16 aa, Artificial
  FVIII fragment
DVHSGLIGPLLVCHTN

SEQ ID NO: 15 - protein, 14 aa, Artificial
  FVIII fragment
SNKCQTPLGMASGH

SEQ ID NO: 16 - protein, 19 aa, Artificial
  FVIII fragment
TPVVNSLDPPLLTRYLRIH

SEQ ID NO: 17 - protein, 16 aa, Artificial
  FVIII fragment
RMKNNEEAEDYDDDLT

SEQ ID NO: 18 - protein, 13 aa, Artificial
  FVIII fragment
EDWDYAPLVLAPD

SEQ ID NO: 19 - protein, 16 aa, Artificial
  FVIII fragment
YPHGITDVRPLYSRRL

SEQ ID NO: 20 - protein, 19 aa, Artificial
  FVIII fragment
LTENIQRFLPNPAGVQLED

SEQ ID NO: 21 - protein, 16 aa, Artificial
  FVIII fragment
RGNSTGTLMVFFGNVD

SEQ ID NO: 22 - protein, 22 aa, Artificial
  FVIII fragment
ETKNSLMQDRDAASARAWPKMH

SEQ ID NO: 23 - protein, 20 aa, Artificial
  FVIII fragment
EAEDYDDDLTDSEMDVVRFD

SEQ ID NO: 24 - protein, 15 aa, Artificial
  FVIII fragment
LLTRYLRIHPQSWVH

SEQ ID NO: 25 - protein, 19 aa, Artificial
  FVIII fragment
SFVNMERDLASGLIGPLLI

SEQ ID NO: 26 - protein, 21 aa, Artificial
  FVIII fragment
SKAGIWRVECLIGEHLHAGMS

SEQ ID NO: 27 - protein, 20 aa, Artificial
  FVIII fragment
QSWVHQIALRMEVLGCEAQD

SEQ ID NO: 28 - protein, 20 aa, Artificial
  FVIII fragment
IQHESGILGPLLYGEVGDTL

SEQ ID NO: 29 - protein, 20 aa, Artificial
  FVIII fragment
LNEHLGLLGPYIRAEVEDNI

SEQ ID NO: 30 - protein, 16 aa, Artificial
  FVIII fragment
EPFSWIKVDLLAPMII

SEQ ID NO: 31 - protein, 23 aa, Artificial
  FVIII fragment
NQDSFTPVVNSLDPPLLTRYLRI

SEQ ID NO: 32 - protein, 20 aa, Artificial
  FVIII fragment
LTRYLRIHPQSWVHQIALRM

SEQ ID NO: 33 - protein, 21 aa, Artificial
  FVIII fragment
ELSWDYMQSDLGELPVDARFP

SEQ ID NO: 34 - protein, 22 aa, Artificial
  FVIII fragment
SGLIGALLVCREGSLAKEKTQT

SEQ ID NO: 35 - protein, 26 aa, Artificial
  FVIII fragment
AQTLLMDLGQFLLFCHISSHQHDGME

SEQ ID NO: 36 - protein, 16 aa, Artificial
  FVIII fragment
YPHGITDVRPLYSRRL

SEQ ID NO: 37 - protein, 19 aa, Artificial
  FVIII fragment
NTLNPAHGRQVTVQEFALF

SEQ ID NO: 38 - protein, 23 aa, Artificial
  FVIII fragment
ATWSPSKARLHLQGRSNAWRPQV

SEQ ID NO: 39 - protein, 25 aa, Artificial
  FVIII fragment
EFLISSSQDGHQWTLFFQNGKVKVF

SEQ ID NO: 40 - DNA, 33 bp, Artificial
  site for library construction
atcaaggccatggcacgannntaaccgcggtaa

SEQ ID NO: 41 - protein, 8 aa, Artificial
  Octapeptide
DYKDDDDK

(19) United States
(12) Patent Application Publication
(10) Pub. No.: US 2010/0055125 A1
(43) Pub. Date: Mar. 4, 2010

(54) Peptide Library

(75) Inventors: Alois Jungbauer, Vienna (AT); Christa Mersich, Vienna (AT)

Correspondence Address: FULBRIGHT & JAWORSKI L.L.P., 600 CONGRESS AVE., SUITE 2400, AUSTIN, TX, 78701, US

(73) Assignee: UNIVERSITAT FUR BODENKULTUR WIEN, Vienna (AT)

(21) Appl. No.: 12/295,251

(22) PCT Filed: Mar. 29, 2007
(86) PCT No.: PCT/AT2007/000148

§ 371 Date: Sep. 29, 2008

(30) Foreign Application Priority Data
Mar. 29, 2006 (AT) A 533/2006

(51) Int. Cl. A61K 39/00; C40B 40/08; C40B 40/02; C12N 1/19; C12N 5/04; C12N 5/00; C40B 50/06; G01N 33/53; C07K 2/00; A61K 38/02; A61P 37/06

(52) U.S. Cl. 424/192.1; 506/17; 506/14; 435/254.2; 435/419; 435/325; 435/254.23; 435/254.21; 506/26; 435/7.2; 436/501; 530/300; 514/2

(57) ABSTRACT

The present invention relates to a vector library comprising a multiplicity of different eukaryotic secretion vectors, wherein each vector comprises under the control of transcriptional and translational control sequences a gene encoding for an extracellular soluble fusion polypeptide which gene comprises a coding sequence for a scaffold polypeptide linked to variable coding sequences for a peptide, wherein said vectors comprise a nucleic acid coding for a secretory signal sequence linked to the gene coding for the fusion polypeptide.

### CROSS-REFERENCE TO RELATED APPLICATIONS

This application is a national phase application under 35 U.S.C. §371 of International Application No. PCT/AT2007/000148 filed 29 Mar. 2007, which claims priority to Austrian Application No. A 533/2006 filed 29 Mar. 2006. The entire text of each of the above-referenced disclosures is specifically incorporated herein by reference without disclaimer.

The present invention relates to vector cell and peptide libraries comprising a multiplicity of different eukaryotic secretion vectors.

### BACKGROUND OF THE INVENTION

1. Field of the Invention

The field of biomolecule screening for biologically and therapeutically relevant compounds is rapidly growing. Relevant biomolecules that have been the focus of such screenings include chemical libraries, nucleic acid libraries, and peptide libraries in search for molecules that either inhibit or augment the biological activity of identified target molecules. With particular regard to peptide libraries, the isolation of peptide inhibitors of targets and the identification of formal binding partners of targets has been a key focus.

2. Description of Related Art

For more than a decade, phage display technology has been applied to elucidate protein-protein and protein-peptide interactions. Random peptide libraries have been useful, e.g., to predict and to screen for epitope sequence mimics of unknown ligands.

Libraries of random-sequence polypeptides are valuable sources of novel molecules which possess a variety of powerful biologic activity. Systems allowing a high-throughput screening of novel proteins secreted from a host like yeast are desirable.

In particular, random peptide technologies have been shown to be powerful tools in biological and medical applications, with potential uses in affinity ligand identification, drug design, development of diagnostic markers and vaccine discovery.

Traditionally, peptides have been produced in phage libraries, mainly as fusions of the phage coat proteins pIII and pVIII of the bacteriophage M13. These fusion proteins tolerate rather short inserts (up to 15 amino acids). Surface expression as a means of library production has also been accomplished in Gram-positive and Gram-negative bacteria. Usually, bacterial display is better suited for the production of antibodies and protein fragments than for the creation of random peptide libraries. Yeast surface display of peptide libraries has also been proposed as an alternative way to generate mammalian proteins. Some eukaryotic proteins expressed in E. coli are insoluble, and cannot be incorporated into phage particles; instead, these proteins have been fused to cell-surface mating adhesion receptors of yeast for use in library creation. Similar technologies to express peptides on the surface of cells were developed with rhinoviruses and insect viruses.

Choice of a particular platform depends on the importance of library size, biosynthetic capability, and quantitative precision for the particular application envisioned.

In the art several types of biological libraries based on in vitro as well as on in vivo ribosomal synthesis are known.

In vitro transcription/translation systems, for instance, allow to generate very large libraries up to 10¹⁵ by obviating a cell transformation step and the control of screening conditions independent of maintenance of cell viability.

The in vitro method ribosome display involves the preservation of a polypeptide-ribosome-mRNA ternary complex as a genetic unit.

Another system involving puromycin-linked peptide-RNA consists of a covalently linked nucleotide and polypeptide. Covalent RNA-peptide complexes are formed by linkage with puromycin in an in vitro transcription/translation reaction.

To localize the phenotypic effects of a mutated enzyme, in vitro transcription/translation reaction was dispersed in an oil-water emulsion to create aqueous compartments with cellular dimensions.

In vivo library display platforms range from virus particles to whole cells, and include prokaryotic and eukaryotic organisms.

In the course of phage display proteins are displayed as fusions to a phage coat protein, and phage particles are isolated by “panning” against a ligand bound on a solid-phase support. The phages are propagated in E. coli.

The filamentous phage minor coat protein pIII is the most widely used display protein and is present at 3-5 copies per virion.

Further the major capsid protein PVIII of the filamentous phage is used for peptide display.

Less used scaffolds for peptide display are the minor coat protein pVI and the D protein of bacteriophage lambda.

In filamentous phages two systems are used: the polyvalent display (“one-gene system”) and the monovalent display (“two-gene system”). In the polyvalent display, the DNA fragments coding for the peptides are inserted into the phage vector, usually between a particular coat protein and its single peptide. In the monovalent display, the phage genome is modified and the defective phage is termed “phagemid”. A phagemid contains the sequences needed for packing into virions, but does not encode viral genes.

Several fusion protein strategies for the display of relatively short peptides on the surface of Gram-negative bacteria have been described. Peptides of less than 60 amino acid residues can be displayed on the cell surface when fused into surface exposed loops of outer membrane proteins (OMPs) from enteric bacteria.

Extracellular appendages like pili and flagella have also been used successfully for the display of peptides. The FLITRX system, an E. coli display vector, was developed based on the major structural component of the E. coli flagellum FliC.

Construction of random peptide libraries has been accomplished as fusions with a DNA binding protein and as fusions with ubiquitin.

A general advantage of eukaryotic systems is the capacity for high fidelity folding of mammalian extracellular proteins and domains.

The two hybrid system is a genetic method that uses transcriptional activity as a measure of protein-protein interaction. It relies on the modular nature of many site-specific transcriptional activators, which consist of a DNA-binding domain and a transcriptional activation domain. The DNA-binding domain targets the activator to the specific genes that will be expressed, and the activation domain contacts other proteins of the transcriptional machinery to enable transcription. In the two-hybrid system, these two domains of the activator are not covalently linked, they can be brought together by the interaction of any two proteins.

The yeast two-hybrid method has been undergoing continual refinement and extension since its invention, resulting in such variants as reverse two-hybrid, three hybrid and one hybrid.

Because of the yeast two-hybrid method requires nuclear localization and transcriptional activation, testing of secretory or cell-surface proteins is generally not viable in this system.

A cytoplasmatic two-hybrid assay based on ubiquitin was also developed. If the C-terminal fragment of ubiquitin is fused to a reporter gene and co-expressed with the amino terminal fragment, the two halves will reconstitute the native ubiquitin, resulting in the cleavage of the reporter protein.

Some glycosylphosphatidylinositol (GPI) anchored proteins on the yeast cell surface have been used successfully as scaffolds for peptide display in yeast.

Several high-throughput applications with intracellular expression of cDNA libraries in yeast have been reported. For instance, a dual vector system for the expression of a human fetal brain cDNA library in P. pastoris and E. coli is described in the art.

Foreign proteins have been displayed on the surface of insect cells, in occlusion bodies and on the baculovirus surface. Fusion proteins with baculoviral envelope protein gp64, with the pg64 anchor sequence as well as foreign membrane proteins such as the influenza virus hemagglutinin were shown to be targeted to the surface of infected insect cells.

Several eukaryotic RNA viruses permit insertion of short peptides into their native envelope proteins at distinct locations, and have been used for peptide display. Identification of coat protein fusions that do not interfere with the retroviral infectivity, opens the possibility for the development of phage-like methodologies with the benefit of posttranslational modifications.

In particular, human rhinovirus is used for the generation of peptide display libraries.

Upon the growing number of options available for polypeptide libraries and screening, it is important to consider the criteria for choosing an appropriate technology for a given application (see Table 1 below). The criteria for selection of the optimal strategy include: available size of the library, peptide size, biosynthetic capabilities of the system and quantitative discrimination from false screening positives.

TABLE 1
Comparison of different biological peptide
display systems

| | | | | | Mammalian |
|---|---|---|---|---|---|
| | | | | | cell |
| | Ribosome | Phage | Bacterial | Yeast | based |
| Property | display | display | display | display | display |
| Theoretical | 10¹⁵ | <10¹¹ | 10⁹ | 10⁸ | 10⁸ |
| upper | | | | | |
| limit of | | | | | |
| library | | | | | |
| size | | | | | |
| Host | In vitro | Prokaryote | Prokaryote | Yeast | Mammalian |
| expression | | | | cell | cell |
| systems | | | | | |
| Linkers | Non | Viral | Cell | Cell | Cell |
| | covalent | capsid | | | |
| | or | | | | |
| | covalent | | | | |
| Insert size | + | + | − | − | − |
| restriction | | | | | |
| Folding | − | Nonnative | Nonnative | Native | Native |
| machinery | | | | | |
| Post- | − | − | − | −/+ | + |
| translational | | | | | |
| mod- | | | | | |
| ifications | | | | | |

In the case of phage displayed libraries, biopanning is the method of choice. The target molecule is bound to a plastic surface and aspecific sites are blocked. The display library is then incubated with the target and the bound clones are eluted, amplified and used for further rounds of selection. Target molecules can be immobilized on immunotubes, microplate wells or beads.

Isolation of specific peptide synthesising clones in cell surface displayed systems may be achieved using fluorescence-activated cell sorting (FACS). Cells are incubated with fluorescently labelled target molecules, and those able to bind the target can be separated. Cell sorting can highly enrich the positive clones and can discriminate between clones of different affinity and specificity. Furthermore, it allows screening with the target molecule in solution. In this way no elution is required, avoiding the isolation of clones binding unspecifically to the solid support and also the elution problem of very tightly binding clones. These cells can also get enriched by magnetic particle technology.

The detection of proteins expressed soluble in cells usually requires a lysis step to access the intracellular products. Single colonies get transferred to membranes, lysed and incubated with the target molecule. The detection of bound ligand is usually done with a labeled second ligand.

Another approach for generating peptide libraries was described in the WO 00/20574. Said PCT application relates to the use of scaffold proteins (e.g. green fluorescent protein) in fusion constructs with random and defined peptides and peptide libraries, to increase the cellular expression levels, decrease the cellular catabolism, increase the conformational stability relative to linear peptides, and to increase the steady state concentrations of the random peptides and random peptide library members expressed in cells for the purpose of detecting the presence of the peptides and screening random peptide libraries.

U.S. Pat. No. 6,270,968 relates to a method for providing a DNA sequence from microorganisms which encodes for a polypeptide exhibiting a specific activity. Said method comprises the following steps:

1) PCR amplification of a DNA sequence encoding a polypeptide with an activity of interest with PCR primers having a homology to known genes encoding said peptide,

2) linking the PCR product to a structural gene,

3) expressing the obtained hybrid DNA sequence,

4) screening for a hybrid DNA sequence encoding polypeptide exhibiting the activity of interest.

In JP 11308993 a process for constructing a cDNA library is described. Said cDNA library allows to identify unknown polypeptides comprising a specific signal peptide.

Prior art, however, lacks powerful and reliable expression systems in eukaryotes, especially yeast, which allow expression and extracellular providement of a peptide library. For many uses, such an extracellular eukaryotic library system would be advantageous compared to present systems which either require lysis of cells or detachment of the members of the peptide library from the extracellular surface of a host organism (or host virus).

### SUMMARY OF THE INVENTION

It is an object of the present invention to provide means and methods for the manufacturing of improved eukaryotic peptide libraries which may be employed for screening purposes.

Therefore the present invention relates to a vector library comprising a multiplicity of different eukaryotic secretion vectors, wherein each vector comprises under the control of transcriptional and translational control sequences a gene encoding for an extracellular soluble fusion polypeptide which gene comprises a coding sequence for a scaffold polypeptide linked to variable coding sequences for a peptide, wherein said vectors comprise a coding sequence for a secretory signal peptide linked to the gene coding for the fusion polypeptide.

In conventional methods (bacterial display, phage display, yeast display, mammalian cell display) the peptide is presented on a surface. Thus reaction partners may be sterically hindered and a different interaction may take place in the surface displayed system than in free solution. The supernatant of our secretory system can be directly used for screening purposes. The combinatorial peptide can be directly subjected to an in-vitro assay. The conventional biopanning does not allow the application of an invasive assay; an assay in which the biomolecule (phage, cell, etc.) is destroyed like in mass spectrometry, electrophoresis or immunological assays using denaturing conditions.

As used herein, the term “extracellular soluble fusion polypeptide” refers to fusion polypeptides which do not bind to the cell wall or cell membrane of a host in which said polypeptides are expressed and secreted. Consequently, if the fusion polypeptide according to the present invention is expressed and secreted it will not remain associated with the cell wall and/or cell membrane of the host cell but will e.g. diffuse into the culture broth or supernatant of the cell culture, and can therefore be considered as a “free” polypeptide.

The term “linked to” referring to coding sequences (i.e. nucleic acids) encoding a polypeptide or peptide signifies that the coding sequences are covalently bound in frame, optionally linked with a suitable linker sequence.

“Eukaryotic secretion vectors” according to the present invention are vectors to be used in eukaryotic hosts comprising signal sequences allowing to secrete a polypeptide into a culture medium, whereby the secreted polypeptide will not remain bound to the cell wall or cell membrane of the eukaryotic host. The eukaryotic secretion vectors according to the present invention may also be shuttle vectors, which means that such vectors, e.g. plasmids, may be propagated in another organism and the expression occurs in another (e.g. propagation in a prokaryotic organism like Escherichia coli and expression in yeast).

The provision of a secretion signal sequence allows to secrete the fusion polypeptide according to the present invention out of the host cell into the supernatant of the culture medium. Therefore, the isolation of said polypeptides is facilitated because a lysis of the host cell as well as a separation of the polypeptides from the surface of the cells is not required.

The peptide to be fused to the scaffold protein may preferably comprise a maximum of 100 amino acid residues, more preferably a maximum of 80 amino acid residues, even more preferably a maximum of 60 amino acid residues, most preferably a maximum of 40 amino acid residues, in particular a maximum of 20 amino acid residues.

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo; i.e., capable of replication under its own control.

In general, expression vectors containing promoter sequences which facilitate the efficient transcription and translation of the inserted DNA fragment are used in connection with the host. The expression vector typically contains an origin of replication, promoter(s), terminator(s), as well as specific genes which are capable of providing phenotypic selection in transformed cells. The transformed hosts can be fermented and cultured according to means known in the art to achieve optimal cell growth.

A DNA “coding sequence”, as used herein, is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A polyadenylation signal and transcription termination sequence will usually be located 3′ to the coding sequence.

“Transcriptional and translational control sequences” are DNA regulatory sequences, such as promoters, enhancers, polyadenylation signals, terminators, and the like, that provide for the expression of a coding sequence in a host cell. A coding sequence is “under the control” of transcriptional and translational control sequences in a cell when RNA polymerase transcribes the coding sequence into mRNA, which is then trans-RNA spliced and translated into the protein encoded by the coding sequence.

A “signal sequence” is also be included with the coding sequence. This sequence encodes a signal peptide, preferably N-terminal to the polypeptide, that communicates to the host cell and secretes the polypeptide out of the cell. Signal sequences suitably used according to the present invention can be found associated with a variety of proteins native to eukaryotes. A “secretory signal sequence” according to the present is consequently a DNA sequence that encodes a peptide that, as a component of a larger polypeptide, directs the larger polypeptide through a secretory pathway of a cell in which it is synthesized. The larger polypeptide is commonly cleaved to remove the secretory peptide during transit through the secretory pathway.

A library, in particular a biological (peptide) library, comprises generally a pool of microorganisms expressing different polypeptides. Each microorganism carries only one encoding DNA sequence for a certain peptide and represents one clone. Each clone of the library can be propagated and will express the same peptide.

A polypeptide library construction starts with the design of the encoding DNA sequence. The source for this insert can be a pool of chemically synthesized degenerated oligonucleotides, cDNA, genomic DNA fragments or mutagenized specific gene fragments. This library will be constituted by viral particles or by cells.

The next step is the screening of the library against the target molecule. Clones identified as binders to the target substance will be sequenced and their coding regions will be translated into the particular peptide sequences.

The introduction of DNA fragments into an appropriate vector and the transformation into microorganisms require optimized protocols to maximize the cloning efficiency, especially for the construction of large libraries. A cell has been “transformed” with exogenous or heterologous DNA, in particular with members of the vector library according to the present invention, when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a vector or plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

Because the cloning of a DNA fragment requires compatible ends with the vector, the two DNAs must be cut with the same restriction enzymes. The vector DNA must be linearized and purified. A ligation reaction is set up where degenerate DNA fragments are mixed at a molar excess with the vector and are ligated together with the enzyme T4 DNA ligase.

The amount of double stranded DNA fragments required depends on the number of randomized nucleotides and on the expectation of how many times a unique sequence should be represented in the library (library complexity). The other important parameter is the transformation efficiency (number of transformants obtained from 1 μg vector DNA) of the system used. Usually high transformation efficiency in E. coli is obtained with electroporation and is around 10⁹ transformants per μg of supercoiled vector DNA, while the efficiency of a cut-and-religated vector is about 10-100 times less. The ligation mix is used to transform competent E. coli cells in several separated transformations. An aliquot of the transformed cells is grown on solid medium and counted for the calculation of the library complexity. The library can be grown in liquid medium. The plasmids can be harvested and purified to transform the final host organism of the library.

As an alternative to insert double stranded oligonucleotides to the vector, methods based on gap repair (DeMarini, D. J., et al., Biotechniques, 2001. 30:520-3) or ligating the single stranded oligonucleotide to one end of the vector, doing the second strand synthesis by the Klenow fragment of DNA polymerase and a second ligation to the other end of the vector.

To improve the affinity of already isolated peptide ligands, a secondary library can be constructed by introducing either targeted or random mutations. In cassette mutagenesis, the target regions are substituted by a synthetic DNA duplex with the desired mutations. In regional mutagenesis, mutations are introduced by chemical or enzymatic treatments at a controlled rate of alterations per nucleotide, and then the DNA is cloned. Combinatorial mutagenesis replaces a certain amount of amino acids per peptide using the cassette method (Merino, E., et al., Biotechniques, 1992. 12:508-10). Spiked oligonucleotides are synthesized by adding at predetermined positions a particular amount of a mixture of different bases in order to spike the wild type base.

The scaffold polypeptide is preferably a polypeptide which can easily be secreted into the supernatant or into the extra-cellular matrix, hence said polypeptide is preferably absent of transmembrane or cell wall/membrane binding domains

The secretability of polypeptides and proteins depends mainly on the physico-chemical properties of the molecules and cannot be predicted from a primary protein sequence except when transmembrane domains are present. Proteins with transmembrane domains are generally not secreted. The secretion can be determined by conventional assays like ELISA, Western Blot, enzymatic tests, etc. A secretim rate satisfying the needs of the method according to the present invention can be found, exemplified from HSA and eIF5a, e.g. in Schuster M et al. (J. Biotechn. 84 (2000) 237-248).

According to a preferred embodiment of the present invention the eukaryotic secretion vector is a yeast, mammalian, insect or plant vector.

It is especially preferred that the secretion vector is suited for protein expression in yeast.

The secretory signal sequence is preferably selected from the group consisting of alpha factor secretion signals listed herein.

According to the present invention all secretory signal sequences known in the art may be suitably used in the vector library provided that they are recognized by the eukaryotic host and induce secretion of the polypeptide fused thereto.

TABLE 2
Overview of secretory signal sequences

Signal Sequence
Yeast mating factor α
Yeast invertase suc2 leader
Yeast acid phosphatase pho1 leader
Yeast acid phosphatase pho5 leader
Yeast inulinase inu1p leader
Yeast α-Galactosidase leader
Yeast killer toxin leaders
K28 killer virus pptox leader
Plant chitinase leader
Synthetic prepro leaders
Native prepro sequence of protein

Proteins destined for secretion preferably feature a signal peptide at the N-terminus.

According to another preferred embodiment of the present invention the yeast vector is selected from the group consisting of YEpFLAG-1, pYES, pYC, p427-TEF, p417CYC, pTEF-MF, pGAL-MF, pESC-HIS, pESC-LEU, pESC-TRP, pESC-URA.

The YEpFLAG-1 vector (Sigma, Mo.) is a 7205 bp yeast expression vector for cloning and extracellular expression of proteins as an N-terminal FLAG fusion protein in the S. cerevisiae BJ3505, host strain. Transcription is regulated from the yeast alcohol dehydrogenase promoter (ADH2) by glucose repression. The promoter is tightly repressed when the yeast host, transformed with a YEpFLAG-1 vector construct, is grown in the presence of glucose. When glucose in the medium is depleted by yeast metabolism, the promoter is derepressed to a high level. The alpha-factor leader sequence encodes an 83 amino acid peptide responsible for extracellular secretion of the yeast alpha-factor mating pheromone. Removal of the leader sequence occurs during extracellular secretion from the BJ3505 host by proteolytic cleavage. This generates a FLAG fusion protein with a free N-terminus. The FLAG epitope (DYKDDDDK—SEQ ID NO: 41), an acidic and highly hydrophilic octapeptide with a high surface probability, allows immunological detection and affinity purification of the fusion protein. The protease deficient yeast strain BJ3505 (pep4::HTS3 prb-delta 1.6R HIS3 lys2-208 trp1-delta 101 ura3-52 gal2 can1) is used for extracellular expression of proteins and allows growth selection on media lacking tryptophan.

Preferred vectors to be used according to the present invention are YEp Vectors. The YEp yeast episomal plasmid vectors replicate autonomously because of the presence of a segment of the yeast 2 μm plasmid that serves as an origin of replication (2 μm ori). The 2 μm ori is responsible for the high copy-number and high frequency of transformation of YEp vectors.

YEp vectors contain either a full copy of the 2 μm plasmid, or, as with most of these kinds of vectors, a region which encompasses the ori and the REP3 gene. The REP3 gene is required in cis to the ori for mediating the action of the trans-acting REP1 and REP2 genes which encode products that promote partitioning of the plasmid between cells at division. Therefore, the YEp plasmids containing the region encompassing only ori and REP3 must be propagated in cir⁺ hosts containing the native 2 μm plasmid.

Most YEp plasmids are relatively unstable, being lost in approximately 10-2 or more cells after each generation. Even under conditions of selective growth, only 60% to 95% of the cells retain the YEp plasmid.

The copy number of most YEp plasmids ranges from 10-40 per cell of cir⁺ hosts. However, the plasmids are not equally distributed among the cells, and there is a high variance in the copy number per cell in populations.

Several systems have been developed for producing very high copy-numbers of YEp plasmids per cell, including the use of the partially defective mutation leu2-d, whose expression is several orders of magnitude less than the wild-type LEU2⁺ allele. The copy number per cell of such YEp leu2-d vectors range from 200-300, and the high copy-number persists for many generations after growth in leucine-containing media without selective pressure. The YEp leu2-d vectors are useful in large-scale cultures with complete media where plasmid selection is not possible. The most common use for YEp plasmid vectors is to overproduce gene products in yeast.

Other preferred vectors used according to the present invention are YIp Vectors. The YpI integrative vectors do not replicate autonomously, but integrate into the genome at low frequencies by homologous recombination. Integration of circular plasmid DNA by homologous recombination leads to a copy of the vector sequence flanked by two direct copies of the yeast sequence. The site of integration can be targeted by cutting the yeast segment in the YIp plasmid with a restriction endonuclease and transforming the yeast strain with the linearized plasmid. The linear ends are recombinogenic and direct integration to the site in the genome that is homologous to these ends. In addition, linearization increases the efficiency of integrative transformation from 10- to 50-fold.

Other vectors preferably used according to the present invention are YCp vectors. The YCp yeast centromere plasmid vectors are autonomously replicating vectors containing centromere sequences, CEN, and autonomously replicating sequences, ARS. The YCp vectors are typically present at very low copy numbers, from 1 to 3 per cell, and possibly more, and are lost in approximately 10⁻² cells per generation without selective pressure. In many instances, the YCp vectors segregate to two of the four ascospore from an ascus, indicating that they mimic the behavior of chromosomes during meiosis, as well as during mitosis. The ARS sequences are believed to correspond to the natural replication origins of yeast chromosomes, and all of them contain a specific consensus sequence. The CEN function is dependent on three conserved domains, designated I, II, and III; all three of these elements are required for mitotic stabilization of YCp vectors. YRp vectors, containing ARS but lacking functional CEN elements, transform yeast at high frequencies, but are lost at too high a frequency, over 10% per generation, making them undesirable for general vectors.

Preferably used Yeast replicative plasmids are YRp vectors able to multiply as independent plasmids because they carry a chromosomal DNA sequence that includes an origin of replication.

According to a preferred embodiment of the present invention the coding sequence for the peptide is linked to the 3′ end of the scaffold polypeptide. Secretion signal may be placed at the N-terminus of protein.

Linking the coding sequence of the peptide to the 3′ end of the coding region for the scaffold polypeptide results in a nucleic acid encoding a preferred fusion polypeptide having fused to its C-terminus a peptide. Of course it is also possible to link the coding sequence of the peptide to the 5′ end of the scaffold polypeptide. In such a case the peptide has to be positioned at the 3′ end of the secretory signal sequence. Furthermore, it may preferably also be possible to link the coding region of the peptide to the 3′ as well as to the 5′ end of the coding sequence of the scaffold polypeptide. Of course the peptides linked to said 3′ and 5′ end may vary or be identical. The resulting fusion polypeptide comprises consequently the peptide at the N- and/or C-terminal of the scaffold polypeptide.

The coding sequence for the peptide encodes preferably for a random or semi-random peptide sequence or is a fragment of a genomic, gene, EST or mRNA nucleic acid molecule.

According to the present invention all kind of peptides may be fused to the scaffold polypeptide. These peptides may be encoded by genomic, gene, EST or mRNA nucleic acid molecules or fragments thereof or be random or semi-random peptide sequences. A vector library comprising said nucleic acid molecules may be used, e.g., for expressing a peptide library which may be used to investigate protein-peptide interactions.

Regarding to random peptide sequences it is noted that in chemical libraries, for instance, the diversity is given by 20ⁿ (20 is the number of different amino acids, n is the number of randomized positions). For example, a complete library constituted of five amino acids will have 3.2×10⁶ different molecules. The longer the peptide sequences are the more error prone the synthesis will be. In chemical libraries there is no bias toward specific amino acids, whereas in biological libraries some amino acids are more represented than others because of the codon degeneracy.

In a fully degenerated oligonucleotide library the diversity is given by (4×4×4)ⁿ, whereas 4 is the number of different nucleotides and n is the number of randomized codons. The size of biological libraries is mainly limited by the transformation efficiency in microorganisms and the amount of cells that can be handled. The upper limit of the transformation efficiency in E. coli is described as 10⁹ transformants per 1 μg vector DNA. Biological libraries can be made of long random polypeptides. If the randomized amino acid positions total more than seven, the library is incomplete (e.g. seven randomized amino acids result in 1.3×10⁹ peptides). Some amino acids will not be evenly distributed because some amino acids are coded by more than one triplet. Other peptides may be toxic for the cell or may be expressed less efficiently. The advantage of long random sequences expressed in incomplete libraries is the fact, that in most cases the binding region is limited to a few amino acid residues. Since a long variable peptide will contain within its sequence several short peptide sections, the total number of different short peptides will be higher than the number of different clones representing the library. Furthermore, long random sequences allow affinity selection or peptide ligands that require the interaction of few residues spaced apart, or small structural elements.

In a fully degenerated oligonucleotide, each triplet will code for one of the 64 possible codons. At each coupling reaction an equal mixture of all four nucleotides (N) will be used for all three positions in the triplet. In this way, the oligonucleotide will contain all 64 possible codons, and all 20 amino acids and three stop codons will be represented.

To avoid certain stop codons or amino acids, some positions of the oligonucleotide cannot be fully randomized. For one position of the triplet a mixture of only two nucleotides will be used instead of a mixture of all four (see the following table 2).

TABLE 3
Design of oligonucleotides

| Triplet | Function | Reference |
|---|---|---|
| NNK | all 20 amino acids | [1, 2] |
| | possible | |
| | only 1 stop codon | |
| | possible | |
| NNS | all 20 amino acids | [1, 2] |
| | possible | |
| | only 1 stop codon | |
| | possible | |
| NNY + RNN | no stop codon | [3] |
| | possible, but Cys | |
| | and Gln missing | |
| RNN + NNG + NHY | Cys missing | [4] |

N = A, C, G, T; K = G, T; S = G, C; Y = C, T; R = A, G, H = A, C, T;
[1] Scott, J. K. and G. P. Smith, Science, 1990. 249 (4967): 386-90;
[2] Smith, G. P. and J. K. Scott, Methods Enzymol, 1993. 217: 228-57;
[3] Mandecki, W., Protein Eng, 1990. 3: 221-6;
[4] Scalley-Kim, M., Protein Sci, 2003. 12: 197-206.

Another way to design randomized oligonucleotides was presented by LaBean et al. (Protein Sci, 1993. 2:1249-54). This method minimizes stop codons and matches amino acid frequencies observed in 207 natural proteins. With the use of a refining-grid search algorithm, termination codons are minimized and amino acid compositions of the peptides get balanced. Three mixtures of nucleotides are designed, each corresponding to one of the three positions in the codon.

A different approach for the synthesis of randomized DNA was described by Neuner et al. (Nucleic Acids Res, 1998. 26:1223-7). The strategy is based on the use of dinucleotide phosphoramite building blocks within a resin-splitting procedure. Seven dinucleotide building blocks are required to encode all the 20 natural amino acids.

There is also a way to constrain peptides by introducing two codons for cysteine in both sides of the random region. The screening of pools of these cyclic libraries (CX5C, CX6C, CX7C) resulted in the isolation of ligands to several integrins (Koivunen, E., et al. Biotechnology (NY), 1995. 13:265-70). Cyclic and linear peptide libraries were also employed to screen for streptavidin binders. The analysis of the binding peptides showed that the conformationally constrained cyclic peptides bound streptavidin three orders of magnitude better than linear peptides (Giebel, L. B., et al., Biochemistry, 1995. 34:15430-5). The usage of split inteins (Scott, C. P., et al., Chem Biol, 2001. 8:801-15) also allows the production of cyclic peptides.

Methods of making randomly sheared genomic DNA and/or cDNA, and of manipulating such DNA's, are also known in the art. (See, e.g., Sambrook et al., Molecular Cloning, A Laboratory Manual, 3rd ed., Cold Spring Harbor Publish., Cold Spring Harbor, N.Y. (2001); Ausubel et al., Current Protocols in Molecular Biology, 4th ed., John Wiley and Sons, New York (1999); which are incorporated by reference herein.) The details of library construction, manipulation and maintenance are also known in the art. (See, e.g., Ausubel et al., supra; Sambrook et al., supra.)

By “randomized” herein is meant that each nucleic acid and peptide consists of essentially random nucleotides and amino acids, respectively. As is more fully described below, the nucleic acids which give rise to the peptides are chemically synthesized, and thus may incorporate any nucleotide at any position. Thus, when the nucleic acids are expressed to form peptides, any amino acid residue may be incorporated at any position. The synthetic process can be designed to generate randomized nucleic acids, to allow the formation of all or most of the possible combinations over the length of the nucleic acid, thus forming a library of randomized nucleic acids. “Semi-randomized”, as used herein, refers to a peptide sequence which is derived from a distinct sequence and wherein single amino acid residues are exchanged by random sequences (e.g. 10%, 30% or 60% of the overall amino acid residues are exchanged).

The library according to the present invention comprises preferably a multiplicity of different eukaryotic secretion vectors of at least 2, preferably at least 10, more preferably of at least 100, most preferably of at least 1000, in particular of at least 10000.

According to a preferred embodiment of the present invention the scaffold polypeptide is a eukaryotic initiation factor, preferably eukaryotic initiation factor 5a (eIF5a), in particular human eukaryotic initiation factor 5a (eIF5a).

The scaffold polypeptide which may be used in the vector/peptide library may exhibit various features including the ability to be secreted efficiently and correctly folded from a host in order to guarantee the accessibility of the peptide according to the present invention to binding partners intended to bind to said peptide.

It is especially preferred to use as scaffold polypeptide eukaryotic initiation factor, preferably eukaryotic initiation factor 5a (eIF5a), in particular human eukaryotic initiation factor 5a (eIF5a). The eukaryotic initiation factor 5a (eIF5a) is a protein essential for survival of the eukaryotic cell. EIF5a is a small (17 kDa) protein which is involved in the first step of peptide-bond formation in translation and it also takes part in the cell-cycle regulation. It is the only known cellular protein to contain the post-translationally derived amino acid hyposine [N^{ε}-(4-amino-2-hydroxybutyl)lysine].

eIF5a is preferably used as scaffold polypeptide because it is ubiquitously expressed in mammals, in particular in humans, and does therefore not show any immune response when administered to said mammals. Furthermore Schuster et al. (Schuster, M., et al., J Biotechnol, 2000. 84:237-48) could show a high yield expression of eIF5a as FLAG fusion product in high yield and purity with the YepFLAG-1 vector system.

Another aspect of the present invention relates to a cell library comprising host cells containing the vector library according to the present invention.

The vector library according to the present invention may be introduced (e.g. transformed) in host cells leading to the formation of a cell library. Said cell library is able to express those polypeptides which are encoded by the vector library.

Yet another aspect of the present invention relates to a host cell comprising one vector of the vector library according to the present invention.

The host cell, which may also be part of a cell library, is preferably yeast host cells, preferably Pichia pastoris, Hansenula polymorpha or Saccharomyces cerevisiae cells, mammalian host cells or plant host cells.

In particular these host cells are suited for expressing the fusion polypeptides according to the present invention.

Another aspect of the present invention relates to a method of generating a peptide library comprising the steps of:
providing vectors of a vector library according to the present invention,
transferring said vectors into host cells,
isolating hosts cells comprising a single vector,
culturing said host cells under conditions suitable for expression of the fusion polypeptides in a culture medium, and optionally
isolating the expressed fusion polypeptides from the supernatant of said culture medium.

The vectors, in particular the vector library, according to the present invention may be transferred (e.g. transformed) into host cells which may be used to express the fusion polypeptides according to the present invention. After transferring the vectors into the host cells, cells comprising a single vector of the library are isolated (i.e. individualized, singularized). Each of the isolated host cells are cultured in order to express and secrete the fusion polypeptides resulting in a peptide library. Optionally the expressed fusion polypeptide may be isolated from the supernatant of the culture medium. The isolation of said polypeptide may be performed by methods well known in the art (e.g. chromatography).

The peptide library according to the present invention may be used in a pharmaceutical preparation or as a vaccine.

According to a preferred embodiment of the present invention the host cells are yeast host cells, preferably Pichia pastoris, Hansenula polymorpha or Saccharomyces cerevisiae cells, mammalian host cells or plant host cells.

Another aspect of the present invention relates to a method for identifying a peptide with a selected biological activity or with a binding capacity to a binding partner, comprising the steps of:
providing a polypeptide obtainable by a method according to the present invention,
contacting said polypeptide with a target cell or a target molecule, and
assessing the ability of the secreted polypeptide to regulate a biological process in a target cell or to bind to a target molecule.

The peptide library according to the present invention may be used for the identification of peptides which exhibit a biological activity or a binding capacity to a binding partner (e.g. antibody). Said activity or said capacity may be evaluated by contacting at least one member of the peptide library (single members or pools of single members) with a target cell or target molecule. The influence of the polypeptide, in particular of the peptide being fused to a scaffold polypeptide, on the target cell or molecule, is determined.

The target molecule is preferably a protein, in particular an enzyme, a receptor, a matrix protein, a cell skeleton protein, an iron transport protein, a peptide hormone, a glucose transporter, an antigen binding protein, an immunoglobulin, a peptide inhibitor, an oxygen transport protein, a signal transduction protein, a transcription factor or a heat-shock protein.

Another aspect of the present invention relates to a pharmaceutical composition comprising a fusion polypeptide comprising a eukaryotic initiation factor, preferably eukaryotic initiation factor 5a (eIF5a), in particular human eukaryotic initiation factor 5a (eIF5a), fused to a pharmaceutically active peptide.

Human eIF5a is a molecule which is ubiquitously expressed in humans and consequently not recognized as foreign polypeptide by the immune system. Therefore, eIF, in particular eIF5a, is a suitable scaffold polypeptide for the introduction of pharmaceutically active peptides. eIF5a is further advantageous because fusion polypeptides involving eIF5a can easily be manufactured as secretion polypeptides in host cells like yeast.

As used herein “pharmaceutically active peptides” may comprise all peptides known in the art which are known to exhibit a biological activity when administered to a human or animal body. The peptides include also antimicrobial peptides like anti-fungal peptides or an anti-bacterial peptides and peptides like insulin/pro-insulin/pre-pro-insulin or variants thereof, peptide hormones like growth hormone, prolaction, FSH, or variants thereof, or blood clotting factor VII or VIII or variants thereof. The term “pharmaceutically active peptide” also applies for peptides which, if conjugated to eIF, in particular eIF5a, according to the present invention, show—as eIF5a-peptide conjugate—a pharmaceutical effect, but not necessarily as a peptide without conjugative to eIF5a.

Therefore, the present invention also relates to a C-terminally elongated eIF, in particular eIF5a, comprising a C-terminal extension of the naturally occurring eIF sequence.

The composition comprises preferably further at least one pharmaceutically acceptable excipient or carrier.

The pharmaceutical composition may further comprise pharmaceutically acceptable excipients and/or carriers. Suitable excipients and carriers are well known in the art (see e.g. “Handbook of Pharmaceutical Excipients”, 5th Edition by Raymond C. Rowe, Paul J. Sheskey, Sian C. Owen (2005), APhA Publications).

According to a preferred embodiment of the present invention the peptide is fused to the C-terminus of the eukaryotic initiation factor, preferably eukaryotic initiation factor 5a (eIF5a).

Due to the three dimensional structure of eIF5a it is preferred that the peptides according to the present invention are fused to the C-terminus of eIF5a, because at this site accessibility of the peptide can be guaranteed. Of course it is also possible to fuse the peptide to the C-terminus of a (N- or C-terminally) truncated eIF5a.

Another aspect of the present invention relates to a vaccine formulation comprising a fusion polypeptide comprising eukaryotic initiation factor, preferably eukaryotic initiation factor 5a (eIF5a), in particular human eukaryotic initiation factor 5a (eIF5a), fused to an antigenic peptide.

An “antigenic peptide”, as used herein, comprises at least 6 amino acid residues of the amino acid sequence of a full length protein and encompasses an epitope thereof such that an antibody raised against the peptide forms a specific immune complex with the full length protein or with any fragment that contains the epitope. Preferably, the antigenic peptide comprises at least 8 amino acid residues e.g. a peptide being 9-11 amino acids in length, or at least 15 amino acid residues, or at least 20 amino acid residues, or at least 30 amino acid residues. Preferred epitopes encompassed by the antigenic peptide are regions of the protein that are located on its surface.

The antigenic peptide is preferably selected from the group consisting of pathogen antigen, tumour associated antigen, enzyme, substrate, self antigen, organic molecule or allergen. More preferred antigens are selected from the group consisting of viral antigens, bacterial antigens or antigens from pathogens of eukaryots or phages. Preferred viral antigens include HAV-, HBV-, HCV-, HIV I-, HIV II-, Parvovirus-, Influenza-, HSV-, Hepatitis Viruses, Flaviviruses, Westnile Virus, Ebola Virus, Pox-Virus, Smallpox Virus, Measles Virus, Herpes Virus, Adenovirus, Papilloma Virus, Polyoma Virus, Parvovirus, Rhinovirus, Coxsackie virus, Polio Virus, Echovirus, Japanese Encephalitis virus, Dengue Virus, Tick Borne Encephalitis Virus, Yellow Fever Virus, Coronavirus, respiratory syncytial virus, parainfluenza virus, La Crosse Virus, Lassa Virus, Rabies Virus, Rotavirus antigens; preferred bacterial antigens include Pseudomonas-, Mycobacterium-, Staphylococcus-, Salmonella-, Meningococcal-, Borellia-, Listeria, Neisseria-, Clostridium-, Escherichia-, Legionella-, Bacillus-, Lactobacillus-, Streptococcus-, Enterococcus-, Corynebacterium-, Nocardia-, Rhodococcus-, Moraxella-, Brucella, Campylobacter-, Cardiobacterium-, Francisella-, Helicobacter-, Haemophilus-, Klebsiella-, Shigella-, Yersinia-, Vibrio-, Chlamydia-, Leptospira-, Rickettsia-, Mycobacterium-, Treponema-, Bartonella-antigens. Preferred eukaryotic antigens of pathogenic eukaryotes include antigens from Giardia, Toxoplasma, Cyclospora, Cryptosporidium, Trichinella, Yeasts, Candida, Aspergillus, Cryptococcus, Blastomyces, Histoplasma, Coccidioides.

The formulation comprises preferably further a pharmaceutically acceptable excipient or carrier or an adjuvant.

Excipients, carriers and adjuvants to be used in vaccines are well known to the person skilled in the art. See for instance “Vaccine Design: subunit & Adjuvant Approach” by Jessica R. Burdman, Michael F. Powell (Editor), Mark J. Newman (Editor), 1995 (Springer/Kluwer).

According to a preferred embodiment of the present invention a peptide or peptide library as defined by the present invention is fused to the C-terminus of the eukaryotic initiation factor 5a (eIF5a). Of course it is also possible to link the coding sequence of the peptide to the 5′ end of the scaffold polypeptide. In such a case the peptide has to be positioned at the 3′ end of the secretory signal sequence. Furthermore, it may preferably also be possible to link the coding region of the peptide to the 3′ as well as to the 5′ end of the coding sequence of the scaffold polypeptide. Of course the peptides linked to said 3′ and 5′ end may vary or be identical. The resulting fusion polypeptide comprises consequently the peptide at the N- and/or C-terminal of the scaffold polypeptide.

The peptide to be fused to eIF5a may exhibit antigenic properties and may consequently be used for an active vaccination of animals and human individuals. The antigenic peptide may be a known antigen or may be identified by a method according to the present invention using a cell library as described herein.

Cloned as carboxy-terminal extensions of eIF5a, the fusion products were produced at high levels in a microplate scale. As a screening application a model approach is described to find peptides which inhibit binding of autoantibodies to clotting factor VIII. The well characterized monoclonal murine antibody ESH8 was employed as a model antibody directed against FVIII.

### BRIEF DESCRIPTION OF THE DRAWINGS

The present invention is further illustrated by the following figures and examples without being restricted thereto.

FIG. 1 shows the workflow of screening for binders from the secreted library.

FIG. 2 shows the cloning site for library construction (SEQ ID NO: 40).

FIG. 3 shows the screening for binders to ESH8. Dot Blots of the supernatants developed with different antibodies: (A) ESH8; (B) ESH8 in presence of FVIII; (C) anti-FLAG M1; (D): secondary antibody anti-IgG-HRP alone. On position A12: FVIII; on positions F12, G12, H12: eIF5a without C-terminal library.

FIG. 4 shows the alignment of the random peptides with the amino acid sequence of human FVIII. Sequence homologies are shown in boldface. FIG. 4A contains sequences with the following SEQ ID numbers:
011F3 is SEQ ID NO: 1
FVIII aa183-198 is SEQ ID NO: 11
FVIII aa561-579 is SEQ ID NO: 12
FVIII aa717-729 is SEQ ID NO: 13
FVIII aa1865-1880 is SEQ ID NO: 14
FVIII aa2037-2050 is SEQ ID NO: 15
FVIII aa2310-2328 is SEQ ID NO: 16

FIG. 4B contains sequences with the following SEQ ID numbers:
013H4 is SEQ ID NO: 2
FVIII aa355-370 is SEQ ID NO: 17
FVIII aa410-422 is SEQ ID NO: 18
FVIII aa495-511 is SEQ ID NO: 19
FVIII aa606-624 is SEQ ID NO: 20
FVIII aa2135-2150 is SEQ ID NO: 21

FIG. 4C contains sequences with the following SEQ ID numbers:
015A2 is SEQ ID NO: 3
FVIII aa230-252 is SEQ ID NO: 22
FVIII aa361-382 is SEQ ID NO: 23
FVIII aa2320-2334 is SEQ ID NO: 24

FIG. 4D contains sequences with the following SEQ ID numbers:
023D3 is SEQ ID NO: 4
FVIII aa554-572 is SEQ ID NO: 25

FIG. 4E contains sequences with the following SEQ ID numbers:
030C8 is SEQ ID NO: 5
030D1 is SEQ ID NO: 6
FVIII aa2010-2031 is SEQ ID NO: 26
FVIII aa2330-2349 is SEQ ID NO: 27
FVIII aa461-480 is SEQ ID NO: 28
FVIII aa1771-1790 is SEQ ID NO: 29
FVIII aa2085-2101 is SEQ ID NO: 30
FVIII aa2305-2327 is SEQ ID NO: 31
FVIII aa2321-2340 is SEQ ID NO: 32

FIG. 4F contains sequences with the following SEQ ID numbers:
032H4 is SEQ ID NO: 7
FVIII aa30-50 is SEQ ID NO: 33
FVIII aa189-210 is SEQ ID NO: 34
FVIII aa315-340 is SEQ ID NO: 35
FVIII aa495-510 is SEQ ID NO: 36
FVIII aa1880-1898 is SEQ ID NO: 37
FVIII aa2220-2242 is SEQ ID NO: 38

FIG. 4G contains sequences with the following SEQ ID numbers:
034F10 is SEQ ID NO: 10
FVIII aa2278-2302 is SEQ ID NO: 39

FIG. 5 shows SDS-PAGE and Western Blots of the secreted fusion proteins. (A) SDS gel, (B) Western Blot developed with anti-FLAG M1, (C) Western Blot developed with ESH8.

FIG. 6 shows the partial neutralization of the inhibitory activity of ESH8 after addition of culture supernatants from the secreted fusion proteins. By adding ESH8, the FVIII activity of normal plasma was reduced to 23.51% of the initial activity. (A) culture supernatants without any dilution, (B) supernatant diluted 1:10, (C) supernatant diluted 1:100.

FIG. 7 shows the partial neutralization of the inhibitory activity of ESH8 after addition of culture supernatants from the fusion proteins. By adding ESH8, the FVIII activity of normal plasma was reduced to 32.6% of its initial activity.

### DETAILED DESCRIPTION

### Examples

### Example 1

The aim of the present example is the implementation of a system of a secreted random peptide library generated in yeast, that allows a high throughput screening in a microplate scale.

The YEpFLAG-1 Expression System for Yeast (Sigma) enables high throughput production and purification of proteins under physiological conditions. Gene expression is auto induced by the alcohol-dehydrogenase promoter. The yeast mating pheromone alpha-leader sequence upstream of the gene fusion site facilitates secretion of the recombinant protein into the culture supernatant. The N-terminal octapeptide FLAG-tag DYKDDDDK (SEQ ID NO: 41) enables rapid detection of the recombinant protein by a monoclonal antibody (Prickett, K. S., et al. Biotechniques, 1989. 7:580-9). The use of the YEpFLAG-1 vector system to produce the Eukaryotic Initiation Factor 5a (eIF5a; GenBank Accession number M23419) delivered a high yield and purity of the recombinant protein (Schuster, M., et al., J Biotechnol, 2000. 84:237-48; Schuster, M., et al., J Biomol Screen, 2000. 5:89-97). Because of these advantages, the protein eIF5a was chosen as a scaffold for the expression of a C-terminal random peptide library. In order to show the working of this strategy, peptides directed to a monoclonal antibody against FVIII were developed.

Approximately 30% of patients suffering from severe hemophilia A develop antibodies against FVIII which neutralizes the effect of the pro-coagulant activity of intravenously injected FVIII, negating the effects of replacement therapy. Various epitopes on the FVIII molecule are bound by these antibodies, but inhibitors binding to the C2 domain or to the A2 domain of FVIII predominate. Phage display libraries have been used to identify FVIII mimotypes (Villard, S., et al., Blood, 2003. 102:949-52; Villard, S., et al., J Biol Chem, 2002. 277:27232-9; Muhle, C., et al., Thromb Haemost, 2004. 91:619-25).

To evaluate the potency of disrupting the interaction between the FVIII molecule and its inhibitors by peptides derived from a random library according to the present invention secreted in yeast—in order to recover the procoagulant activity of FVIII—a model system was investigated. The murine monoclonal antibody ESH8, a well characterized inhibitor that binds to the C2 domain of FVIII, was employed to screen for potential binding peptides and to characterize their potential to break down the interactions of the inhibitor to FVIII.

In this example the design, construction, expression and screening of a library of random polypeptides secreted into the culture supernatant as fusion products with eIF5a is described. Furthermore, the potency of these derived peptides in restoring pro-coagulant activity plasma preparations incubated with FVIII antibodies was tested.

### Material and Methods

Library Construction

General methods for DNA manipulation in vitro were applied according to Sambrook et. al. (Sambrook, J., et al., Molecular Cloning: A Laboratory Manual. 1989, Cold Spring Harbor, N.Y.: Cold Spring Harbor Laboratory Press).

Randomized peptides were designed using an established reading frame and three mixtures of nucleotides, corresponding to the three codon positions. For the first and the second position of each triplet equal mixtures of all four nucleotides (“N”) were used. The third position had a mixture of dC and dG (“S”). In this way, the mixture would contain only 32 triplets instead of 64, but all 20 amino acids would be represented, and only one termination codon (amber) would be possible. The oligonucleotide inserts were amplified by PCR and purified using a MinElute PCR Purification Kit (Qiagen). The purified random sequences were digested with the restriction enzymes NcoI and Cfr42I (both from MBI Fermentas).

The plasmid YEpFLAG-1 (Sigma) was used as both the cloning and expression vector. In the first step, the gene of eIF5a was inserted between the EcoRI and Cfr42I sites in YEpFLAG-1. In the second step, the random library was inserted between the NcoI site of eIF5a and the Cfr42I site of the plasmid.

The resulting constructs were transformed to competent E. coli cells GeneHogs (Invitrogen) by electroporation. The plasmids were recovered by a Plasmid Preparation Kit (Maxi Kit, Qiagen) and transformed to the yeast strain BJ3505 (Sigma) by a lithium acetate method and grown on plates containing selective Synthetic Complete Medium without tryptophane (Sigma).

Gene Expression, Screening and Protein Characterization

Single clones were transferred to 96-well microplates containing 200 μl Yeast Peptone High Stability Expression Medium (YPHSM) liquid medium. The growth and induction of the yeast cells was performed at 28° C. for 4 days.

The culture supernatants were spotted on Protran nitrocellulose membranes (Schleicher & Schuell) using a Dot-Blot apparatus (Bio-Rad). The membranes were incubated either with the murine anti-FVIII antibody ESH8 (American Diagnostica) or with the anti-FLAG antibody M1 (Sigma). The development of the blots was performed using an anti-mouse-IgG-HRP-conjugate (A-8429, Sigma) and Super Signal West Pico Chemiluminscent Substrate (Pierce). The chemiluminescence signals were detected using a luminescence imager (Boehringer Mannheim).

Clones giving a positive signal were cultivated for a second screening step. The membranes were incubated again with ESH8 and M1 as described in the first screening round; additionally one more membrane was incubated with ESH8 in presence of 10 IU/ml FVIII (Octapharma). Positive clones were evaluated by the intensity of their chemilumenscence signals.

The plasmids from the positive clones were recovered using a Yeast Plasmid Isolation Kit (RPM) and amplified in E. coli. After plasmid purification, sequencing was performed.

For cultivation at a larger scale, overnight cultures of the positive yeast clones were used to inoculate YPHSM in shaker flasks and grown for 3 days at 28° C. The cells were removed by centrifugation at 10,000×g for 5 minutes. The supernatants were immediately frozen and stored at −20° C. For SDS electrophoresis and Western Blotting, the culture supernatants were mixed with 4× NuPage LDS Sample Buffer (Invitrogen) and 0.2 M DTT before freezing.

SDS-PAGE was performed using 4-12% NuPage Novex Bis-Tris gradient gels (Invitrogen) in a Xcell Mini-Cell system (Novex). Gels were stained using GelCode Blue Stain Reagent (Pierce). For Western Blotting, the proteins were transferred to Protran nitrocellulose membranes using the Xcell Mini-Cell system. The development of the blots was performed as described under development of the Dot-Blots.

Fusion protein concentrations were determined by a SPR method. The monoclonal antibody M2 was immobilized by EDC/NHS chemistry on a CM 5 chip (BIACORE). Binding of FLAG fusion proteins generates a response which is proportional to the bound mass. A standard curve using Bacterial Alkaline Phosphatase (Sigma) as reference was used to calculate protein concentrations.

Capacity of Peptides to Neutralize an Antibody Directed Against FVIII

The antibody ESH8 was added to Normal Reference Plasma (American Diagnostica) at a fixed concentration giving 70% activity reduction. After addition of culture supernatants at serial dilutions, the mixtures were incubated for 2 hours at 37° C. The remaining FVIII activity was determined using a Coamatic FVIII Activity Kit (Chromogenix).

Results and Discussion

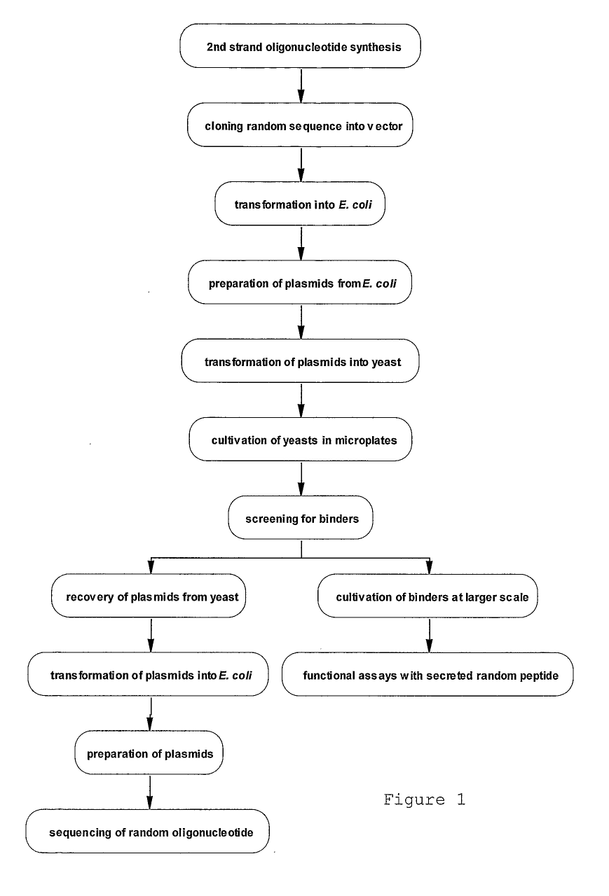

FIG. 1 shows the representation of the workflow of library design, library cultivation, screening and characterization of peptides derived from the library.

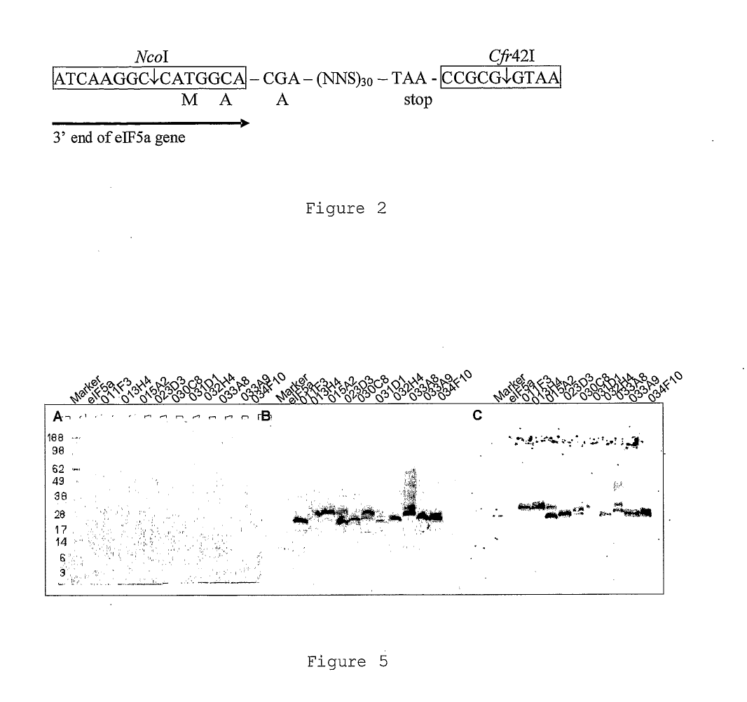

Synthetic oligonucleotides used for library construction contained long variable sections of 30 random codons, flanked on both ends by constant sequences (FIG. 2). Every randomized codon (“NNS”) encoded for all 20 amino acids, 30 codons were set in a line. This would create a random library for peptides with a length of 30 amino acids. At the 3′-terminus of the random sequence a stop codon (ochre) was placed.

To identify peptides binding to ESH8, the culture supernatants of 3,080 single clones cultivated in microplates were screened.

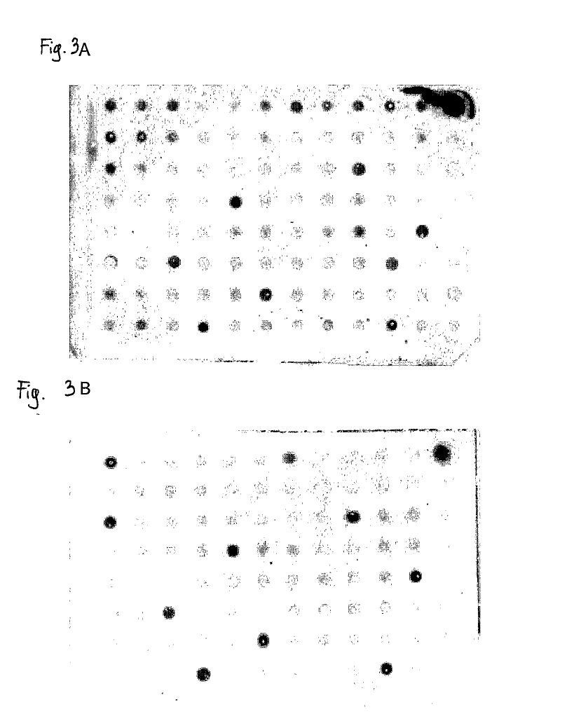

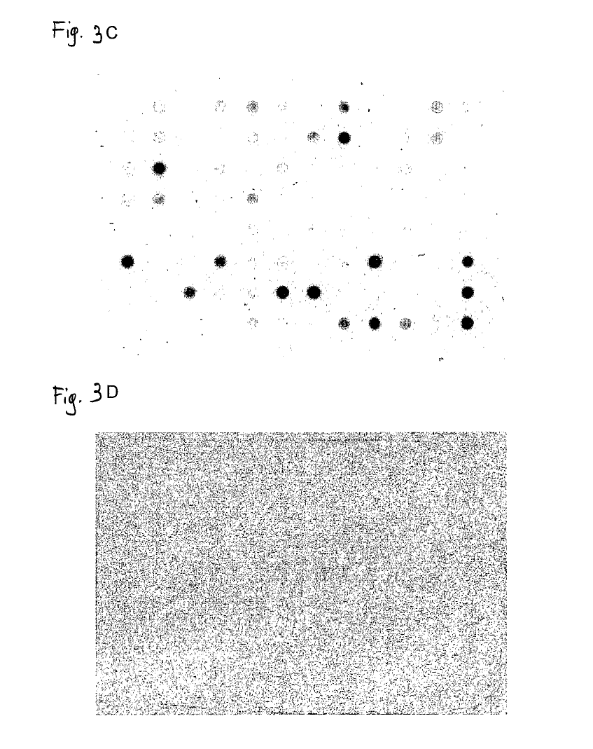

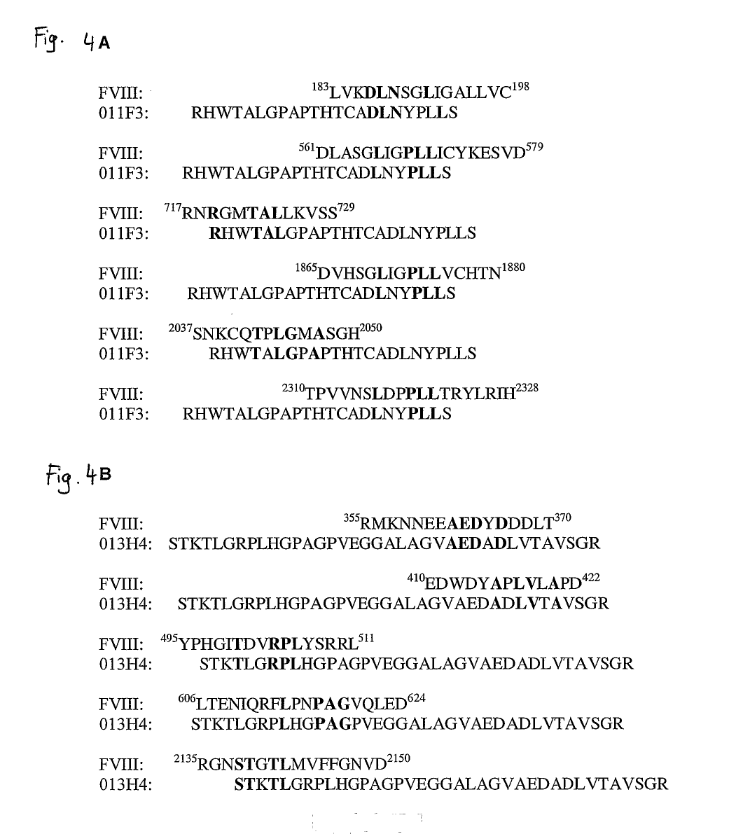

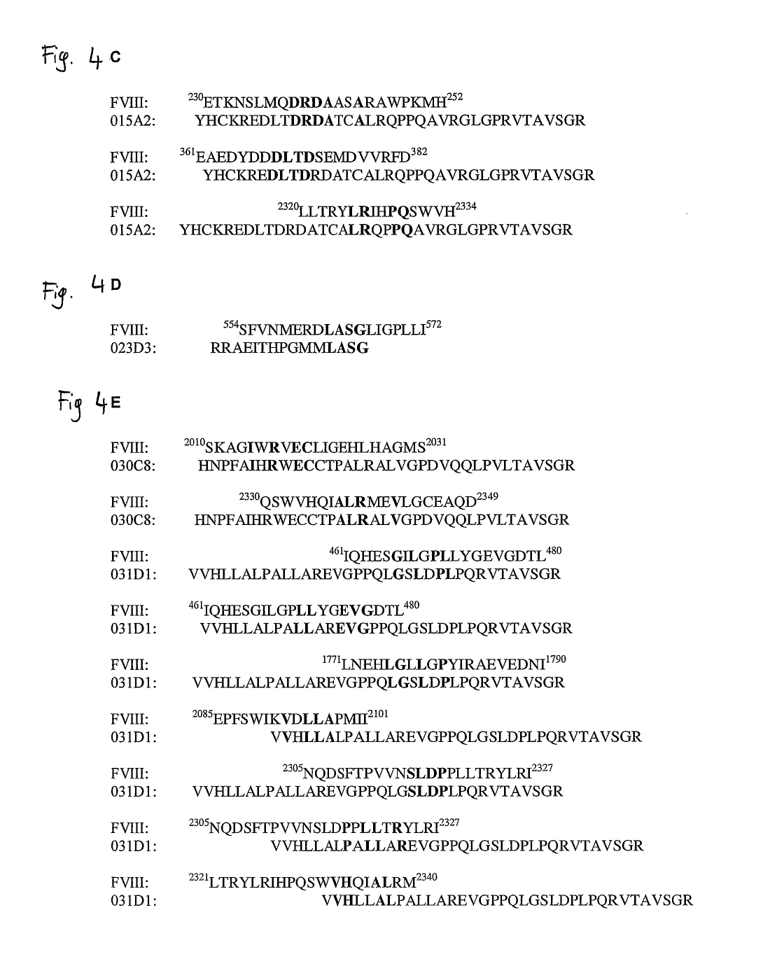

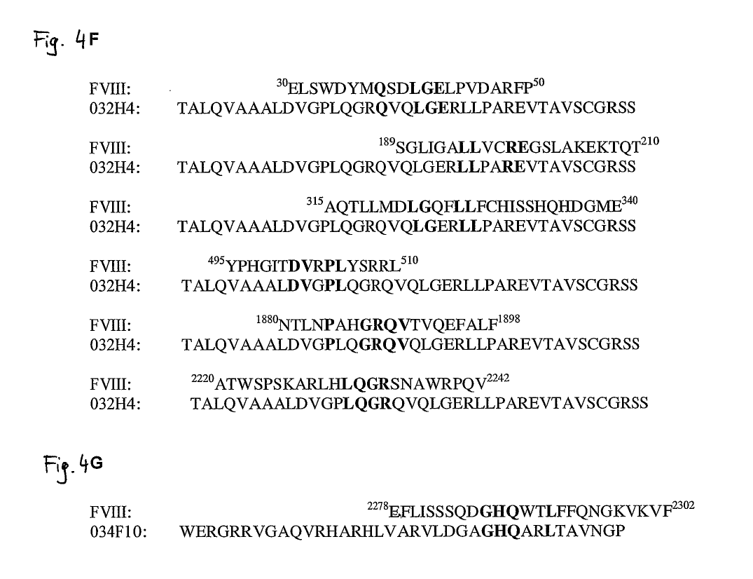

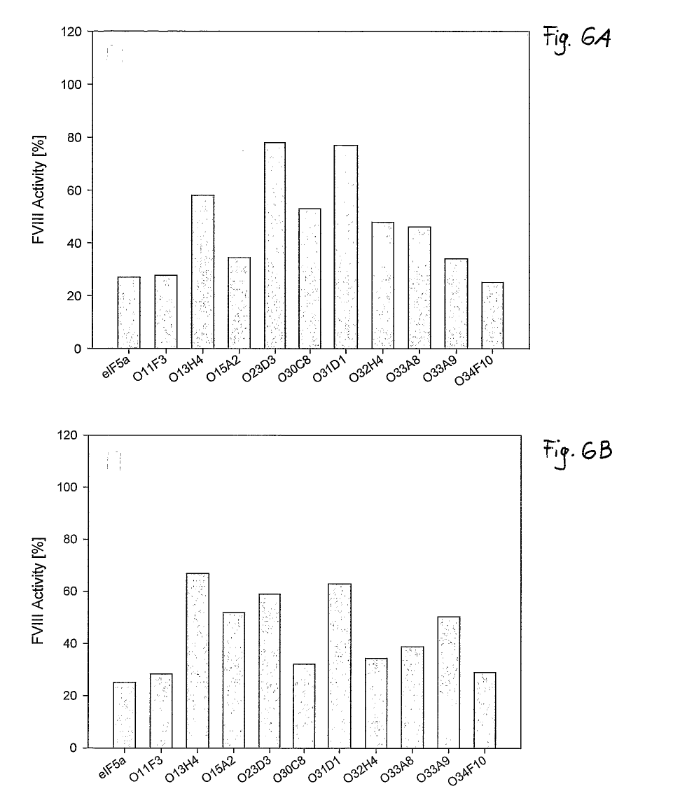

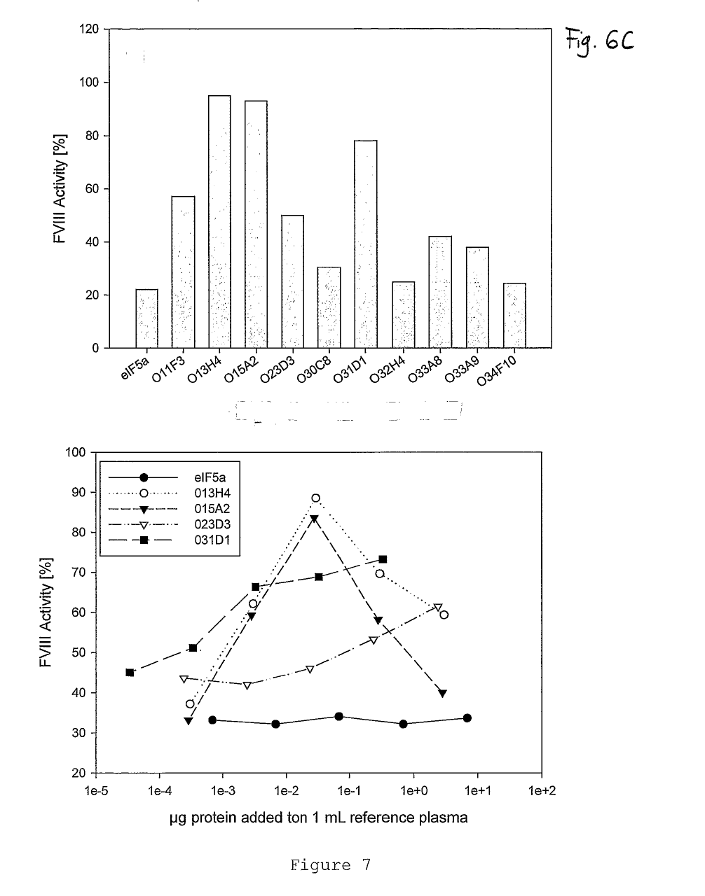

For the second round of screening, 88 clones derived from the first screening round were picked. Their supernatants were spotted on nitrocellulose membranes and incubated with ESH8, ESH8 and FVIII, and the anti-FLAG M1 antibody (FIG. 3). Development was performed with an anti-mouse IgG HRP conjugate. FIGS. 3A and 3B show which secreted proteins bound to ESH8 and to ESH8 in presence of FVIII. Under competitive conditions there were fewer positive dots obtained. The incubation with the anti-FLAG antibody M1 (FIG. 3C) allowed the estimation of the amount of recombinant protein secreted. As negative control, one membrane was incubated with the anti-mouse IgG HRP conjugate alone (FIG. 3D).

Supernatants of clones binding to ESH8 in the presence of FVIII and not binding to the secondary antibody alone were further characterized.

The plasmids of ten positive clones were recovered and sequenced. The peptide sequences and the parameters of the resulting fusion proteins are listed in Table 3. The sequencing results indicate that in some clones the reading frames of the random sequences were corrupted. This could have happened during the oligonucleotide synthesis or during the PCR of the oligonucleotide. These clones synthesized longer peptides than intended. The sequences of the random peptides were compared with the FVIII sequence (FIG. 4). For the peptides 033A8 and 033A9 no sequence homologies could be found. The other peptides showed short consensus sequences with the A1, A2, A3, C1 or C2 domains of FVIII.

TABLE 4

Sequences of the random peptides and
concentration detected in the culture supernatants.

| | | | |
|---|---|---|---|
| | | cprot | Seq ID |
| ID | Peptide Sequence | [ug/ml] | No. |

| | | | |
|---|---|---|---|
| 011F3 | RHWTALGPAPTHTCADLNYPLLS | 29 | 1 |
| 013H4 | STKTLGRPLHGPAGPVEGGALAGVAEDADLVTAVSGR | 36 | 2 |
| 015A2 | YHCKREDLTDRDATCALRQPPQAVRGLGPRVTAVSGR | 34 | 3 |
| 023D3 | RRAEITHPGMMLASG | 29 | 4 |
| 030C8 | HNPFAIHRWECCTPALRALVGPDVQQLPVLTAVSGR | 8 | 5 |
| 031D1 | VVHLLALPALLAREVGPPQLGSLDPLPQRVTAVSGR | 4 | 6 |
| 032H4 | TALQVAAALDVGPLQGRQVQLGERLLPAREVTAVSCGRSS | 20 | 7 |
| 033A8 | NVGTCTSSPARCGWPRRRTSCAALAGLLV | 48 | 8 |
| 033A9 | KADILPEMNSMRADRM | 40 | 9 |
| 034F10 | WERGRRVGAQVRHARHLVARVLDGAGHQARLTAVNGP | 9 | 10 |

In FIG. 5 the SDS-PAGE and Western Blots of the cultivation of the clones at a larger scale are shown.

The different culture supernatants were tested for their capacity to inhibit the interaction of the monoclonal antibody ESH8 with FVIII in a FVIII activity assay. The changes of FVIII activity at a constant concentration of ESH8 were examined in the presence of decreasing amounts of the fusion proteins (see FIG. 6). All specific proteins decreased the inhibitory effects of ESH8 resulting in higher FVIII activities, whereas the addition of the scaffold protein eIF5a alone had no effect on the activity.

The neutralizing properties of proteins 013H4, 015A2, 023D3 and 031D1 were confirmed in another FVIII activity test, where the range of the amount of protein added was broadened. (FIG. 7). The random peptide of clone 031D1—which showed strong inhibition of ESH8 even at low concentrations—shows 3 sequence homologies with the C2 domain of FVIII (see FIG. 4E). The motifs SLDP, P-LL-R, VH-AL of the random peptide can be found in the FVIII sequence from 2315Ser-2338Leu. The motif of peptide 013H4 ST-TL can be found in FVIII at 2138Ser-2142Leu, the motif of 015A2 LR-PQ at 2325Leu—2330Gln. The peptide 023D3 shows no homologies with the C2 domain.

This system provides for a quick and easy method by which long random proteins can be expressed and subsequently screened for new interactions with target proteins. A system for construction of diverse libraries of random-sequence peptides as secreted fusion products with eIF5a was designed and implemented. The over-expression of novel genes regulated by the alcohol-dehydrogenase promoter allowed production of fusion proteins at levels up to 80% of total protein in the supernatant. The yeasts bearing the library were as easily cultivated on a microplate scale as they are in shaker flasks. The screening of this library for binding partners could easily be performed on nitrocellulose membranes.

1.-20. (canceled)

22. A vector library comprising a plurality of different eukaryotic secretion vectors, wherein each vector comprises under the control of transcriptional and translational control sequences a gene encoding for an extracellular soluble fusion polypeptide which gene comprises a coding sequence for a scaffold polypeptide linked to variable coding sequences for a peptide, wherein said vectors comprise a coding sequence for a secretory signal peptide linked to the gene coding for the fusion polypeptide.

23. The library of claim 22, wherein the eukaryotic secretion vector is a yeast, mammalian, insect or plant vector.

24. The library of claim 22, wherein the secretory signal sequence is yeast mating factor α, yeast invertase suc2 leader, yeast acid phosphatase phol leader, yeast acid phosphatase pho5 leader, yeast inulinase inulp leader, yeast α-Galactosidase leader, yeast killer toxin leaders, K28 killer virus pptox leader, plant chitinase leader, synthetic prepro leaders, or native prepro sequence of protein.

25. The library of claim 22, wherein the yeast vector is YEpFLAG-1, pYES, pYC, p427-TEF, p417CYC, pTEF-MF, pGAL-MF, pESC-HIS, pESC-LEU, pESC-TRP, or pESC-URA.

26. The library of claim 22, wherein the coding sequence for the peptide is linked to the 3′ end of the scaffold polypeptide.

27. The library of claim 22, wherein the coding sequence encodes for a random or semi-random peptide sequence or is a fragment of a genomic, gene, EST or mRNA nucleic acid molecule.

28. The library of claim 22, further defined as comprising at least 2 different eukaryotic secretion vectors.

29. The library of claim 28, further defined as comprising at least 10 different eukaryotic secretion vectors.

30. The library of claim 29, further defined as comprising at least 100 different eukaryotic secretion vectors.

31. The library of claim 30, further defined as comprising at least 1,000 different eukaryotic secretion vectors.

32. The library of claim 31, further defined as comprising at least 10,000 different eukaryotic secretion vectors.

33. The library of claim 22, wherein the scaffold polypeptide is a eukaryotic initiation factor (eIF).

34. The library of claim 33, wherein the eukaryotic initiation factor is eukaryotic initiation factor 5a (eIF5a).

35. The library of claim 34, wherein the eukaryotic initiation factor is human eukaryotic initiation factor 5a (eIF5a).

36. A cell library comprising host cells containing a vector library of claim 22.

37. The cell library of claim 36, wherein the host cells are yeast, mammalian, or plant cells.

38. The cell library of claim 37, wherein the host cells are further defined as Pichia pastoris, Hansenula polymorpha, or Saccharomyces cerevisiae cells.

39. A host cell comprising one vector of the vector library of claim 22.

40. The host cell of claim 39, further defined as a yeast, mammalian, or plant cell.

41. The host cell of claim 40, further defined as Pichia pastoris, Hansenula polymorpha, or Saccharomyces cerevisiae cells.

42. A method of generating a peptide library comprising:
providing vectors of a vector library of claim 22;
transferring said vectors into host cells;
isolating hosts cells comprising a single vector; and
culturing said host cells under conditions suitable for expression of the fusion polypeptides in a culture medium.

43. The method of claim 42, further comprising isolating the expressed fusion polypeptides from the supernatant of the culture medium.

44. The method of claim 42, wherein the host cells are yeast, mammalian, or plant cells.

45. The method of claim 44, wherein the host cells are further defined as Pichia pastoris, Hansenula polymorpha, or Saccharomyces cerevisiae cells.

46. A method for identifying a peptide with a selected biological activity or with a binding capacity to a binding partner, comprising:
providing a polypeptide obtainable by a method of claim 42;
contacting said polypeptide with a target cell or a target molecule; and
assessing the ability of the secreted polypeptide to regulate a biological process in a target cell or to bind to a target molecule.

47. The method of claim 46, wherein the target molecule is a protein or a receptor.

48. The method of claim 47, wherein the protein is an enzyme.

49. A pharmaceutical composition comprising a fusion polypeptide comprising a eukaryotic initiation factor fused to a pharmaceutically active peptide.

50. The pharmaceutical composition of claim 49, wherein the eukaryotic initiation factor is eukaryotic initiation factor 5a (eIF5a).

51. The pharmaceutical composition of claim 50, wherein the eukaryotic initiation factor is human eukaryotic initiation factor 5a (eIF5a).

52. The pharmaceutical composition of claim 49, further comprising at least one pharmaceutically acceptable excipient or carrier.

53. The pharmaceutical composition of claim 49, wherein the peptide is fused to the C-terminus of the eukaryotic initiation factor.

54. A vaccine formulation comprising a fusion polypeptide comprising a eukaryotic initiation factor fused to a peptide.

55. The vaccine formulation of claim 54, wherein the eukaryotic initiation factor is eukaryotic initiation factor 5a (eIF5a).

56. The vaccine formulation of claim 55, wherein the eukaryotic initiation factor is human eukaryotic initiation factor 5a (eIF5a).

57. The vaccine formulation of claim 54, further comprising at least one pharmaceutically acceptable excipient or carrier or an adjuvant.

58. The vaccine formulation of claim 54, wherein the peptide is fused to the C-terminus of the eukaryotic initiation factor.
